(19) United States
(12) Patent Application Publication
(10) Pub. No.: US 2011/0196418 A1
(43) Pub. Date: Aug. 11, 2011

(54) FLEXIBLE MEDICAL INSTRUMENTS

(76) Inventor: Salvatore Castro, Raleigh, NC (US)

(21) Appl. No.: 12/511,050

(22) Filed: Jul. 28, 2009

(51) Int. Cl. A61B 17/00

(52) U.S. Cl. 606/205

(57) ABSTRACT

A flexible medical instrument includes an elongate shaft, a jaw tip on the distal end of the elongate shaft and a handle on the proximal end. The elongate shaft includes a coil tube having a distal end fixedly attached to a portion of the jaw tip and a proximal end fixedly attached to a rigid structure at the proximal end of the instrument. The coil tube is disposed within an outer sheath that includes a tubular braid. The elongate shaft and jaw tip are axially rotatable relative to the handle for rotational positioning of the jaws. An actuation element is longitudinally moveable within the shaft to open and close the jaws. The sheath prevents or restricts expansion of the coil tube during pushing of the actuation element.

### FIELD OF THE INVENTION

The present invention relates generally to the field of medical instruments. More particularly, the present invention relates to flexible instruments for use in laparoscopic or endoscopic procedures and which have torque transmissive shafts.

### BACKGROUND

Flexible endoscopic surgical dissectors, graspers, forceps etc are conventional instruments used for endoscopic procedures. In a typical embodiment, the device's jaws are connected to a common clevis pin mounted in a clevis which is coupled to the distal end of a shaft which includes an elongate flexible coil.

Advances in laparoscopic or single port surgical techniques have created new uses for flexible instruments beyond their traditional endoscopic use. For example, Applicant's co-pending U.S. application Ser. No. ______, entitled MULTI-INSTRUMENT ACCESS DEVICES AND SYSTEMS, filed Jul. 29, 2009, Attorney Docket No. TRX-2220, describes a multi-instrument access system in which flexible instruments are introduced into the body via deflectable cannulas. In one type of disclosed system, two such deflectable cannulas are disposed through a single access port in a body wall. The deflectable cannulas have actuators at their proximal ends that are responsive to movement of the handles of the instruments disposed in the cannulas. Thus, even though the instruments are not themselves actively deflectable, the user can cause deflection of the distal ends of the instruments by manipulating their handles.

Conventional flexible instrument suffer from certain drawbacks when used with access technology such as that described in Applicant's co-pending application. The coil gives the shaft of the device the requisite flexibility for use in a flexible endoscope or other flexible, articulating or non-linear access device. However, the coil shaft is, at best, only torqueable in one direction, since the coil structure renders the shaft vulnerable to twisting when the torque is applied to the instrument, thus limiting the user's ability to axially rotate the jaws when needed. Moreover, the flexible proximal end of a conventional instrument shafts lack the ability to operatively couple with the actuators of a deflectable cannula of the type described above in a manner that enables the actuator to translate manipulation of the instrument handle into deflection of the distal ends of the instrument cannulas.

A third drawback, which is found in conventional dissector designs, lies in their inability to deliver adequate forces both when the jaws are being opened to spread tissue and when the jaws are being closed to grasp tissue. With conventional graspers, the pull-wire is pulled to move the jaws to one position (opened or closed), and the pull-wire is pushed to move the jaws to the other position. Pulling the pull-wire places the springs in compression and allows adequate force transmission to the tissue using the jaws. However, when the pull-wire is moved forward, the coils of the spring can separate, thereby reducing the amount of force delivered to tissue by the jaws as they are moved to the closed or opened position.

Instruments disclosed in the present application constitute improvements over conventional flexible instruments due to their torsional rigidity, their ability to operatively couple to actuation devices of the type described in the co-pending application, and their ability to deliver adequate forces at the jaws (e.g. dissection forces) when opened and closed.

### BRIEF DESCRIPTION OF THE DRAWINGS

FIG. 1 is a perspective view of an embodiment of a forceps device;

FIG. 2 is a perspective view of the distal end of the device of FIG. 1 in which features of the shaft are partially exploded;

FIG. 3 is a perspective view of the distal end of the shaft with the outer sheath removed;

FIG. 4 is an exploded perspective view of the jaw tip;

FIG. 5 is a perspective view of the jaw tip with the clevis exploded from the other components;

FIG. 6A is a partially exploded view of the proximal portion of the shaft of the device of FIG. 1;

FIG. 6B is a cross-section view of the shaft taken along the plane designed 6B-6B in FIG. 1;

FIG. 7 is a longitudinal cross-section view of the proximal portion of the shaft and the handle.

FIG. 8 is a perspective view of an embodiment of a second embodiment in the form of a grasping forceps device;

FIG. 9 is a perspective view of the distal end of the device of FIG. 8 in which features of the shaft are partially exploded;

FIG. 10 is similar to FIG. 9, but shows only the clevis exploded from the other components. The outer tube is not shown in FIG. 10;

FIG. 11A is a partially exploded view of the proximal portion of the shaft of the device of FIG. 8;

FIG. 11B is a cross-section view of the shaft taken along the plane designed 11B-114B in FIG. 8;

FIG. 12 is a longitudinal cross-section view of the proximal portion of the shaft and the handle.

### DETAILED DESCRIPTION

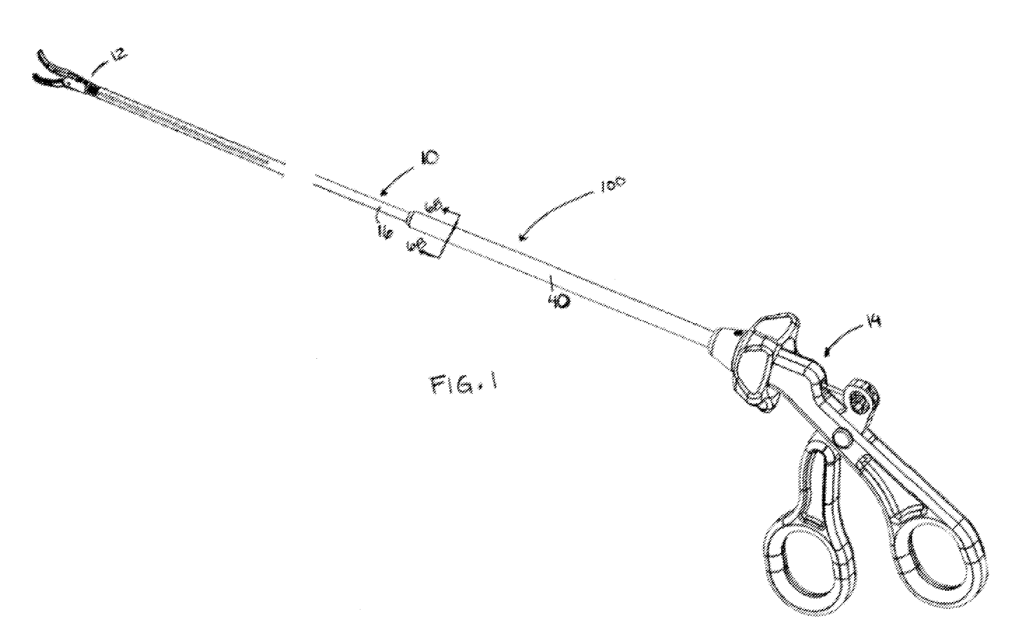

FIG. 1 shows an embodiment of a medical instrument 100. The instrument 100 includes an elongate shaft 10 having a Maryland dissector jaw tip 12 at its distal end. A handle 14 is mounted to the proximal end of the elongate shaft 10.

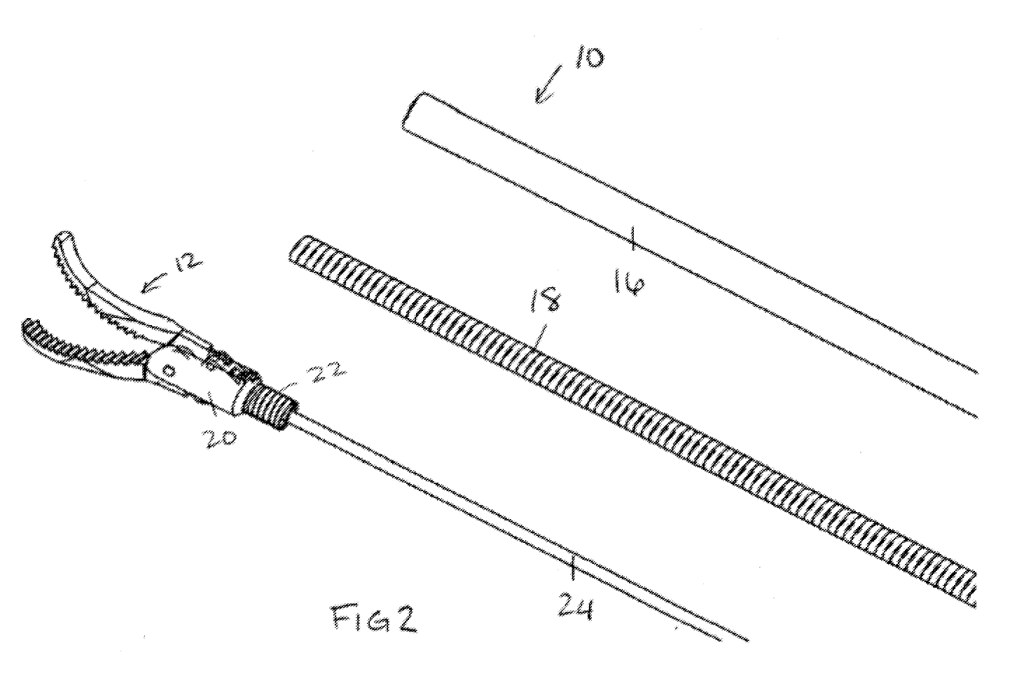

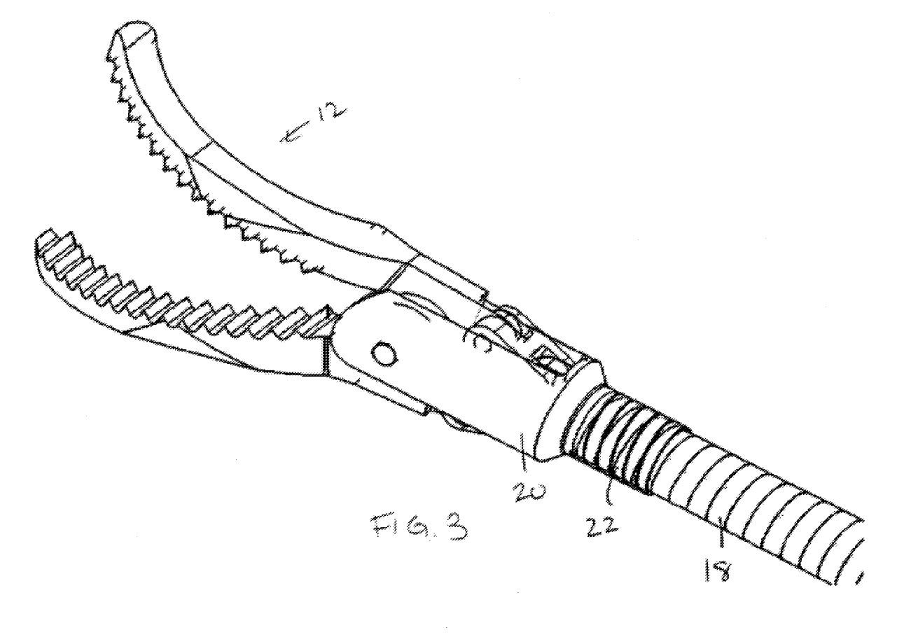

Referring to FIG. 2, the elongate shaft 10 includes an elongate coil 18 extending distally from the handle 14. The coil is preferably formed of a stainless steel ribbon having a rectangular cross-section. A clevis 20 having a tubular collar 22 is positioned at the distal end of the shaft 10. The tubular collar 22 of the clevis is disposed over and fixedly attached to the distal end of the coil tube 18 (also see FIG. 3) such as by welding or other methods. A composite flexible outer sleeve 16 is positioned over the tubular collar 22 and the coil tube 18 and extends to the proximal end of the shaft 10. The outer sleeve 16 is a torque transmissible sleeve formed of a polymeric inner layer (e.g. PTFE), a polymeric outer layer (e.g. Pebax), and a braid between the inner and outer layers. The braid is preferably a kink-resistant stainless steel flat wire braid woven in a diamond pattern, although other patterns might alternatively be used. The outer sleeve 16 is fixed to the tubular collar 22 of the clevis by bonding or other suitable methods.

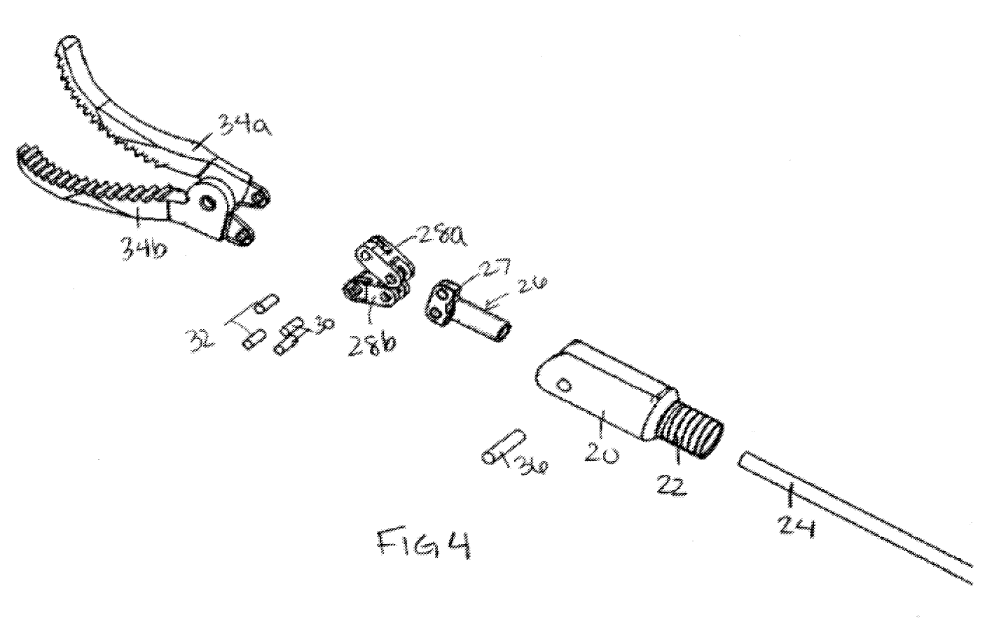

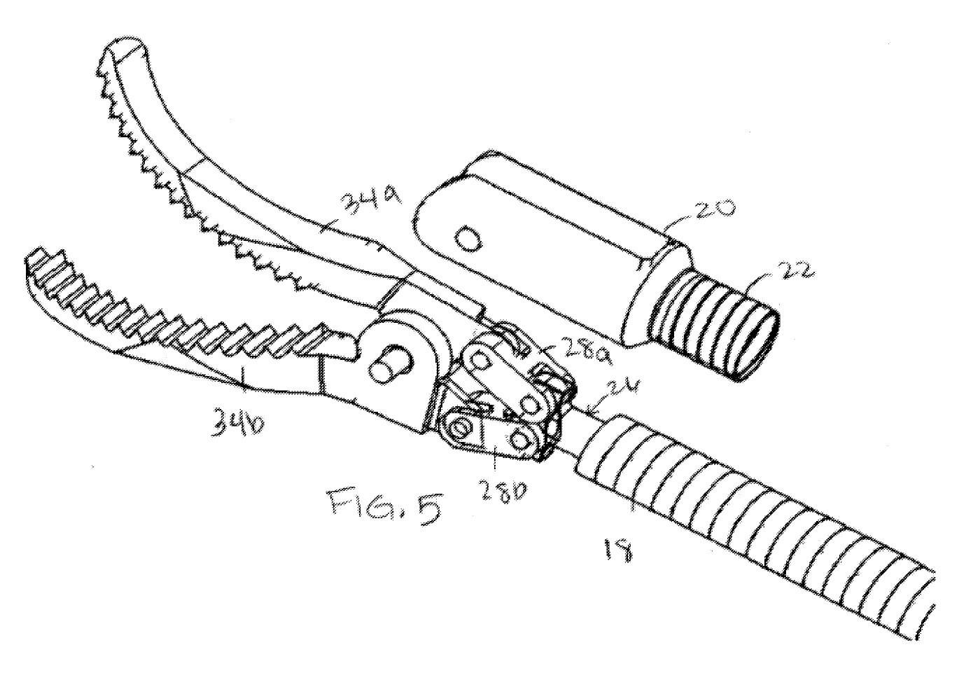

The actuation cable 24 extends from the lumen of the coil 18 into the clevis 20. Referring to FIG. 4, the actuation cable 24 includes an actuation tip 26 having a head 27 pivotally coupled to upper and lower jaw links 28a, 28b by proximal pins 30. Distal pins 32 couple jaw members 34a, 34b of the jaw tip 12 to the links 28a, 28b. The jaw members 34a, 34b are pivotable about a common clevis pin 36 that extends through holes 38 in the clevis 20. With the illustrated arrangement of jaw links and jaw members, movement of the actuation cable 24 in a proximal direction moves the jaws from a closed position to the open position shown in the drawings. Movement of the actuation cable 24 in a distal direction closes the jaws. The jaws may be biased in the opened or closed position, or they may be unbiased.

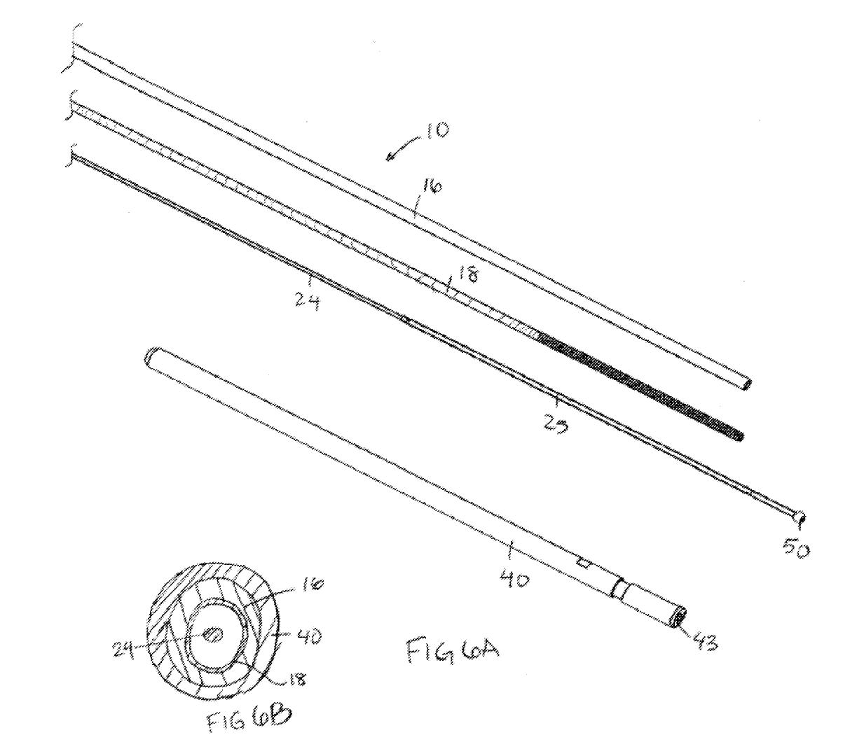

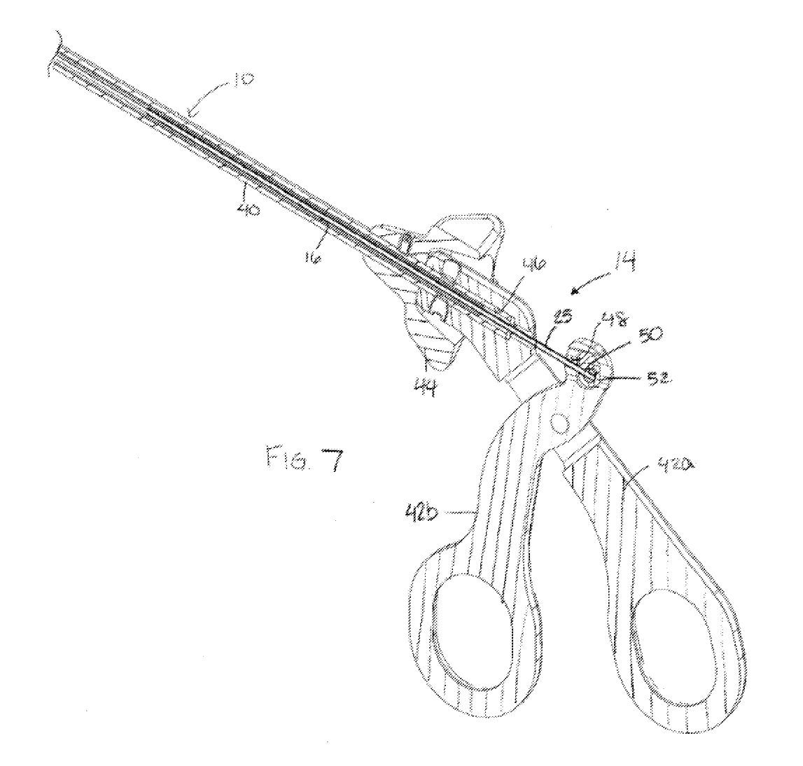

Referring to FIGS. 6A and 6B, a rigid tube 40 is disposed around a proximal portion of the outer tube 16 proximal to the handle 14 and is attached to the outer tube by bonding or other methods. An end plate such as a washer 43 (FIG. 4A) is attached at the proximal end of the instrument tube 40. The coil tube 18 is fixedly attached to the instrument tube 40 at the end plate 43. As shown in FIG. 7, the handle includes a pair of grips 42a, 42b pivotally coupled to one another. A knob 44 rotatably positioned on a distal portion of the handle 14 (e.g. at grip 42a) is fixed to the rigid tube 40 such that rotation of the knob results in axial rotation of the rigid tube, outer tube 16 and coil. The rigid tube 40, the outer tube 16, and the inner coil (not shown in FIG. 7) comprising the proximal portion of the shaft 10 extend proximally from the knob 44 into a bore 46 in the grip 42a of handle 14. The shaft 10 is rotatable within the bore 46 when it is axially rotated by the knob 44.

As best shown in FIG. 6B, the actuation cable 24 extends proximally into the lumen of a rigid actuation tube 25 disposed within the rigid tube 40. The actuation cable 24 is welded, soldered, or otherwise fixed rigid tube. The actuation tube 25 extends out of the rigid tube 40 through washer 43 as most easily seen in FIG. 7, and is disposed within an opening 48 in the grip 42b. An element 50 (e.g. the bead 50 shown in FIG. 6B) at the proximal end of the actuation tube 25 is captured within the opening 48 in a manner that permits the cable to axially rotate with the shaft 10 is rotated. In the drawings, the element 50 is captured within a pivot 52 in the grip 42b.

To close the jaws, the user squeezes the grips 42a, 42b, causing the lower portion of grip 42b to pivot towards grip 42a. The upper portion of the 42b pivots proximally, pushing the actuation tube 25 and cable 24 distally. Because the proximal and distal ends of the coil 18 are fixed to the outer tube 16, the ability of the coil 18 to stretch when the actuation tube/cable 25/24 are pushed is limited by the ability of the outer tube 16 to stretch. Thus, the outer tube 16 is constructed to be significantly more stretch resistant than the coil. The material forming the outer tube 16 is selected to give the outer tube a durometer that will resist stretching sufficiently allow the jaws to close with the closing forces adequate for the procedure. For any given coil, a higher durometer shaft will allow higher closing forces to be achieved at the jaws, whereas a lower durometer shaft will stretch more readily and will thus limit the closing forces that can be achieved at the jaws.

To open the jaws, the grip 42b is pivoted forward to pull the actuation tube 25 and cable 24 proximally, thereby spreading the jaws to the opened position.

To change the axial orientation of the jaws, the user rotates the knob 44, causing the rigid tube 40, inner coil 18, outer tube 16 and actuation cable/tube 24/25 to rotate axially. The braid of the outer tube as well as the rigid coupling of the coil to both the clevis and the rigid tube provide torqueability with a substantially one to one ratio of movement between the handle and the jaw tip regardless of the direction in which the instrument is being axially rotated.

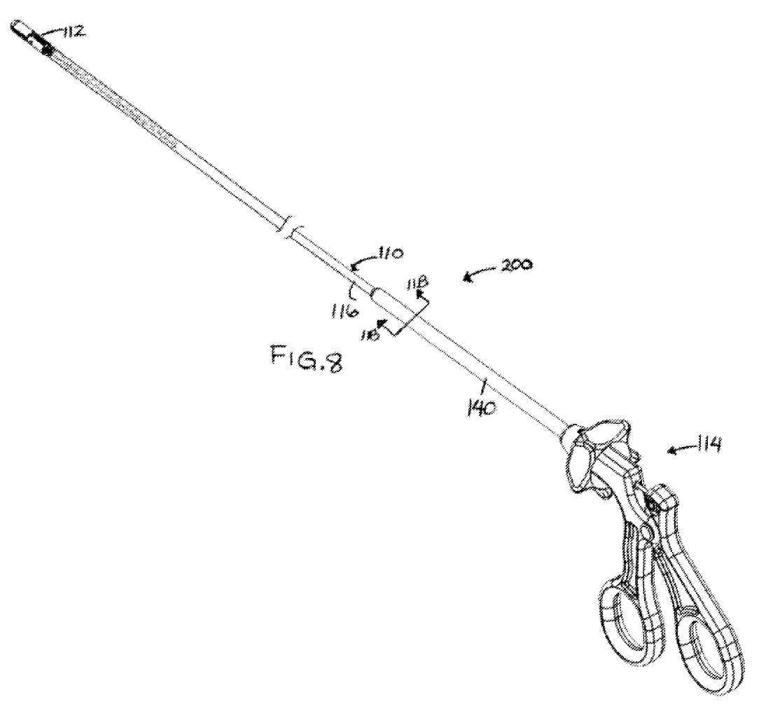

It should be appreciated that the principles of the invention may be applied to other forms of devices that utilize jaws, such as graspers, scissors, clip appliers and forceps, as wells as instruments such as electrocautery hooks that do not have jaws. FIG. 8 shows an alternative embodiment of a medical instrument 200 in the form of a blunt nose grasper.

The instrument 200 includes an elongate shaft 10 having a jaw tip 112 at its distal end. A handle 114 is mounted to the proximal end of the elongate shaft 110.

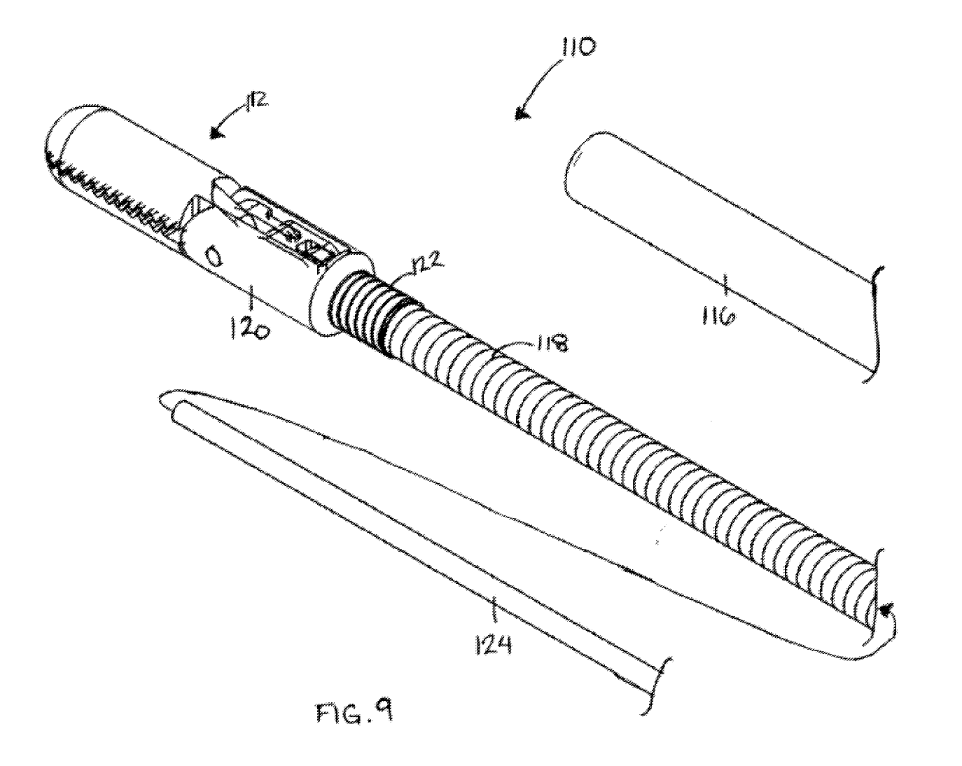

Referring to FIG. 9, the elongate shaft 10 includes an elongate coil 18 extending distally from the handle 14. The coil is preferably formed of a stainless steel ribbon having a rectangular cross-section. A clevis 120 having a tubular collar 122 is positioned at the distal end of the shaft 110. The tubular collar 122 of the clevis is fixedly attached to the distal end of the coil tube 118 such as by welding or other methods. A composite flexible outer sleeve 116 is positioned over the tubular collar 122 and the coil tube 18 and extends to the proximal end of the shaft 110. The outer sleeve 116 is a torque transmissible sleeve formed of a polymeric inner layer (e.g. PTFE), a polymeric outer layer (e.g. Pebax), and a braid between the inner and outer layers. The braid is preferably a kink-resistant stainless steel flat wire braid woven in a diamond pattern, although other patterns might alternatively be used. The outer sleeve 16 is fixed to the tubular collar 122 of the clevis by bonding or other suitable methods.

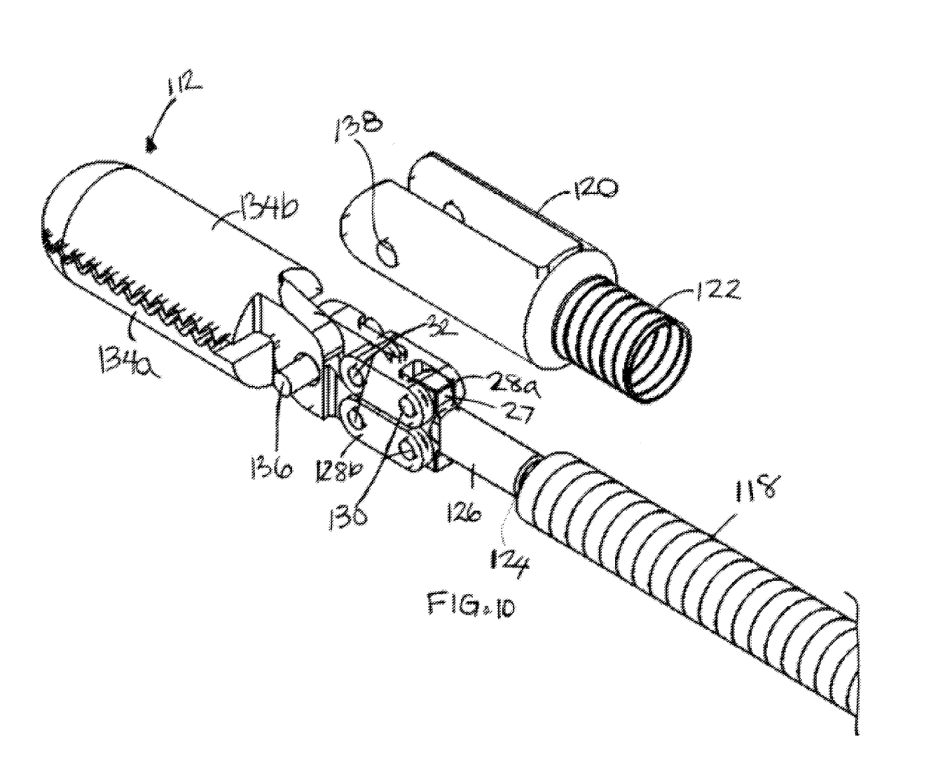

The actuation cable 124 extends from the lumen of the coil 118 into the clevis 120. Referring to FIG. 10, the actuation cable 24 includes an actuation tip 126 having a head 127 pivotally coupled to upper and lower jaw links 128a, 128b by proximal pins 130. Distal pins 132 couple jaw members 134a, 134b of the jaw tip 112 to the links 128a, 128b. The jaw members 134a, 134b are pivotable about a common clevis pin 136 that extends through holes 138 in the clevis 120. With the illustrated arrangement of jaw links and jaw members, movement of the actuation cable 124 in a proximal direction moves the jaws from an opened position to the closed position shown in FIG. 9. Movement of the actuation cable 124 in a distal direction opens the jaws. The jaws may be biased in the opened or closed position, or they may be unbiased, depending on the application for the instrument.

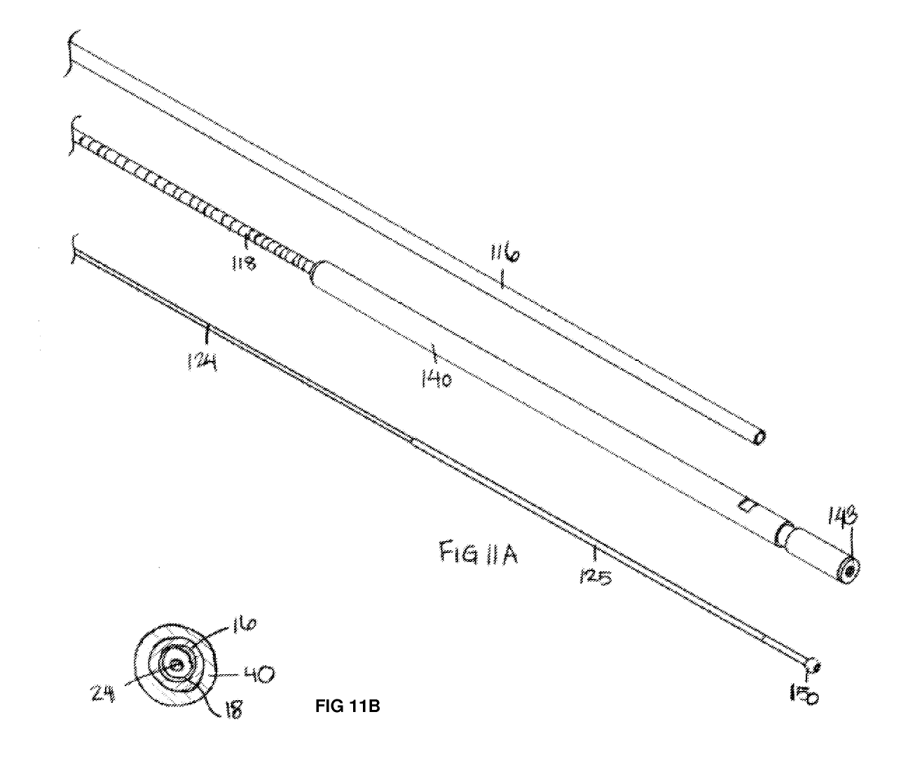

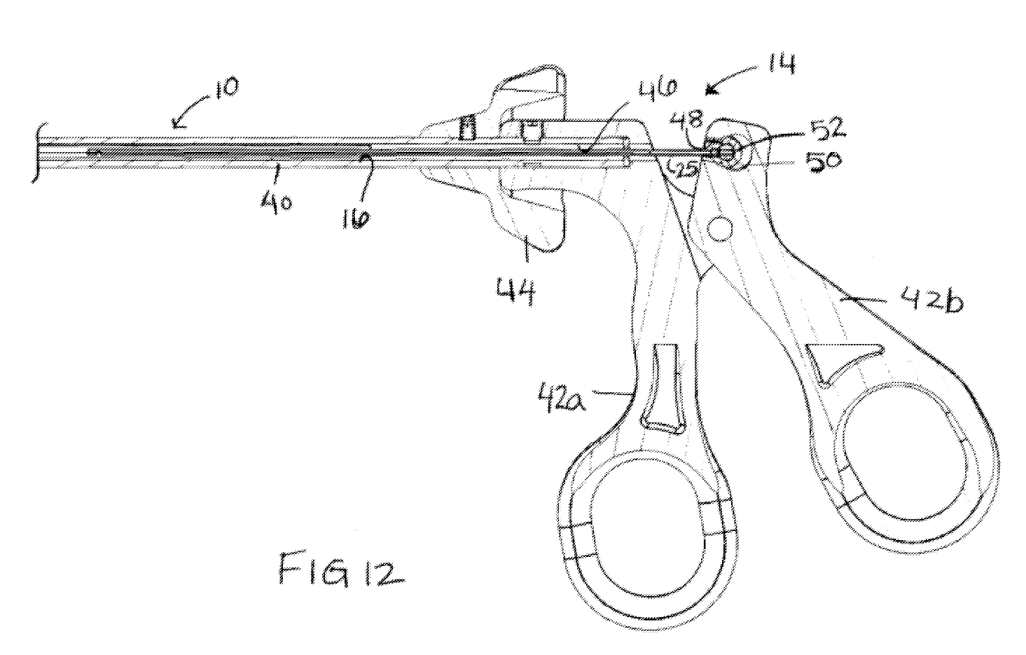

Referring to FIGS. 11A and 11B, a rigid tube 140 is disposed around a proximal portion of the outer tube 116 proximal to the handle 114 and is attached to the outer tube by bonding or other methods. An end plate such as a washer 143 (FIG. 11A) is attached at the proximal end of the instrument tube 140. The coil tube 118 is fixedly attached to the instrument tube 140 at the end plate 143. As shown in FIG. 12, the handle includes a pair of grips 142a, 142b pivotally coupled to one another. A knob 144 rotatably positioned on a distal portion of the handle 114 (e.g. at grip 142a) is fixed to the rigid tube 140 such that rotation of the knob results in axial rotation of the rigid tube, outer tube 116 and coil. The rigid tube 40, the outer tube 116, and the inner coil (not shown in FIG. 12) comprising the proximal portion of the shaft 110 extend proximally from the knob 144 into a bore 146 in the grip 142a of handle 114. The shaft 110 is rotatable within the bore 146 when it is axially rotated by the knob 144.

As best shown in FIG. 11B, the actuation cable 124 extends proximally into the lumen of a rigid actuation tube 125 disposed within the rigid tube 140. The actuation cable 124 is welded, soldered, or otherwise fixed rigid tube. The actuation tube 125 extends out of the rigid tube 140 through washer 143 as most easily seen in FIG. 12, and is disposed within an opening 148 in the grip 142b. An element 150 (e.g. the bead 150 shown in FIG. 11B) at the proximal end of the actuation tube 125 is captured within the opening 148 in a manner that permits the cable to axially rotate with the shaft 110 is rotated. In the drawings, the element 150 is captured within a pivot 152 in the grip 142b.

To close the jaws, the user squeezes the grips 142a, 142b, causing the lower portion of grip 142b to pivot towards grip 142a. The upper portion of the 142b pivots proximally, pulling the actuation cable 124 proximally. To change the axial orientation of the jaws, the user rotates the knob 144, causing the rigid tube 140, inner coil 118, outer tube 116 and actuation cable/tube 124/125 to rotate axially. The braid of the outer tube as well as the rigid coupling of the coil to both the clevis and the rigid tube provide torqueability with a substantially one to one ratio of movement between the handle and the jaw tip regardless of the direction in which the instrument is being axially rotated.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed.

Any and all patents, patent applications and printed publications referred to above are incorporated by reference.

We claim:

1. A flexible dissector comprising:
a rigid tube;
an elongate flexible shaft disposed in the rigid tube, a portion of the shaft extending distally from the rigid tube, the shaft including
a tubular coil having a proximal end and a distal end, the proximal end fixed to a proximal portion of the rigid tube,
a tubular outer sleeve disposed over the coil, the tubular outer sleeve comprising a braid; and
a jaw tip fixed to the distal end of the coil, the jaw tip including a pair of jaws moveable between an opened position and a closed position;
an actuating element extending through the coil and operatively coupled to the jaws, the actuating element longitudinally moveable relative to the elongate flexible shaft between proximal and distal positions;
wherein the outer tubular sleeve is configured to restrict longitudinal expansion of the coil during movement of the actuating element to the distal position.

2. The medical instrument of claim 1, wherein the jaw tip includes a clevis, the clevis affixed to the distal end of the coil.

3. The medical instrument of claim 1, wherein the rigid tube includes a proximal plate on its proximal end, and wherein the coil is affixed to the proximal plate.

4. The medical instrument of claim 2, wherein:
the jaw tip includes a clevis;
the clevis is affixed to the distal end of the coil;
the rigid tube includes a proximal plate on its proximal end;
the coil is affixed to the proximal plate; and
the medical instrument further includes an actuation element disposed within the coil, the medical instrument having a first end coupled to the jaws and having a second end coupled to a handle.

5. The medical instrument of claim 2 further including a handle positioned at a proximal end of the shaft, the actuation element having a first end coupled to the jaws and a second end coupled to the handle such that manipulation of the handle moves the jaws between opened and closed positions.

6. The medical instrument of claim 5, wherein the actuation element includes a flexible distal portion and a rigid proximal portion.

7. The medical instrument of claim 6, wherein the rigid proximal portion of the actuation element is at least partially disposed within the rigid tube.

8. The medical instrument of claim 1, wherein the outer sleeve further includes a polymeric outer layer covering the braid.

9. The medical instrument of claim 2, wherein the clevis includes a tubular collar having a lumen, and wherein the outer surface of the coil is attached to the surface of the lumen.

10. The medical instrument of claim 9, wherein a distal portion of the outer sleeve is positioned over the tubular collar.

11. The medical instrument of claim 10, wherein an inner surface of the outer sleeve is attached to the outer surface of the tubular collar.

12. A method of using a flexible medical instrument, comprising;
introducing a flexible medical instrument into a patient, the flexible medical instrument including a tubular coil having a proximal end and a tubular outer sleeve disposed over the coil, and connected to the proximal and distal ends of the coil, the tubular outer sleeve comprising a braid; and a jaw tip fixed to the distal end of the coil, the jaw tip including a pair of jaws moveable between first and second positions;
pulling an actuating element extending through the shaft and coupled to the jaws, wherein pulling the actuating element moves the jaws to the first position;
pushing the actuating element coil to move the jaws to the second position, wherein the outer tubular sleeve restricts longitudinal expansion of the coil during pushing of the actuating element.

13. The method of claim 12 wherein the first position in an opened position and wherein the second position is a closed position.

14. The method of claim 13 wherein the first position is a closed position and wherein the second position is an opened position.

15. A medical instrument comprising:
a flexible tubular coil having a proximal end and a distal end,
a jaw tip coupled to the distal end of the coil, the jaw tip including a pair of jaws moveable between an opened position and a closed position;
an actuating element extending through the coil and operatively coupled to the jaws, the actuating element longitudinally moveable relative to the elongate flexible shaft between proximal and distal positions; and
a tubular outer sleeve disposed over the coil, the tubular outer sleeve fixedly coupled to the proximal and distal ends of the tubular coil so as to restrict longitudinal expansion of the coil during movement of the actuating element to the distal position.

16. The medical instrument of claim 1, wherein the jaw tip includes a clevis, the clevis affixed to the distal end of the coil.

17. The medical instrument of claim 16, wherein the clevis includes a tubular collar having a lumen, and wherein the outer surface of the coil is attached to the surface of the lumen.

18. The medical instrument of claim 15 further including a handle positioned at a proximal end of the coil, wherein the actuation element has a first end coupled to the jaws and a second end coupled to the handle and wherein manipulation of the handle moves the actuation element
between the proximal and distal position for movement of the jaws.

19. A medical instrument comprising:
a rigid tube;
an elongate flexible shaft disposed in the rigid tube, a portion of the shaft extending distally from the rigid tube, the shaft including
a tubular coil having a proximal end and a distal end, the proximal end fixed to a proximal portion of the rigid tube,
a tubular outer sleeve disposed over the coil, the tubular outer sleeve comprising a braid; and
an end effector fixed to the distal end of the coil.

20. The medical instrument of claim 19, wherein the end effector includes a jaw tip having a clevis and a pair of jaws, the clevis affixed to the distal end of the coil.

21. The medical instrument of claim 19, wherein the rigid tube includes a proximal plate on its proximal end, and wherein the coil is affixed to the proximal plate.

22. The medical instrument of claim 20, wherein:
the end effector includes a jaw tip having a clevis and a pair of jaws;
the clevis is affixed to the distal end of the coil;
the rigid tube includes a proximal plate on its proximal end;
the coil is affixed to the proximal plate; and
the medical instrument further includes an actuation element disposed within the coil, the actuation element having a first end coupled to the jaws and having a second end coupled to a handle.

23. The medical instrument of claim 20 further including a handle positioned at a proximal end of the shaft and an actuation element having a first end coupled to the jaws and a second end coupled to the handle such that manipulation of the handle moves the jaws between opened and closed positions.

24. The medical instrument of claim 23, wherein the actuation element includes a flexible distal portion and a rigid proximal portion.

25. The medical instrument of claim 24, wherein the rigid proximal portion of the actuation element is at least partially disposed within the rigid tube.

26. The medical instrument of claim 19, wherein the outer sleeve further includes a polymeric outer layer covering the braid.

27. The medical instrument of claim 20, wherein the clevis includes a tubular collar having a lumen, and wherein the outer surface of the coil is attached to the surface of the lumen.

28. The medical instrument of claim 27, wherein a distal portion of the outer sleeve is positioned over the tubular collar.

29. The medical instrument of claim 28, wherein an inner surface of the outer sleeve is attached to the outer surface of the tubular collar.
